(19) United States
(12) Patent Application Publication
(10) Pub. No.: US 2024/0292837 A1
(43) Pub. Date: Sep. 5, 2024

(54) CHROMONE DERIVATIVES AS ATTRACTANTS AND REPELLENTS OF BLOOD-SUCKING, BITING INVERTEBRATES

(71) Applicants: CENTRE NATIONAL DE LA RECHERCHE SCIENTIFIQUE, PARIS (FR); CPE LYON, VILLEURBANNE CEDEX (FR); INSTITUT NATIONAL DE LA SANTE ET DE LA RECHERCHE MEDICALE (INSERM), PARIS (FR); INSTITUT DE RECHERCHE POUR LE DÉVELOPPEMENT, MARSEILLE CEDEX 02 (FR); UNIVERSITE CLAUDE BERNARD LYON 1, VILLEURBANNE CEDEX (FR); UNIVERSITE DE LA REUNION, SAINT DENIS (FR); TROPICAL BIOCONTROL, SAINT-DENIS (FR)

(72) Inventors: Mbolatiana Tovo ANDRIANJAFY, ANTANANARIVO (MG); Voahangy VESTALYS, ANATANANARIVO (MG); Marc LEMAIRE, VILLEURBANNE (FR); Patrick MAVINGUI, SAINTE CLOTILDE (FR); Fenia Diane RAMIHARIMANANA, ANTANANARIVO (MG); Saholy LAMARE, SAINT-DENIS (FR)

(21) Appl. No.: 18/261,799

(22) PCT Filed: Jan. 17, 2022
(86) PCT No.: PCT/EP2022/050882

§ 371 Date: Jul. 17, 2023

(30) Foreign Application Priority Data
Jan. 19, 2021 (FR) 2100502

(51) Int. Cl. A01N 43/16; A01P 17/00; A01P 19/00

(52) U.S. Cl. CPC A01N 43/16; A01P 17/00; A01P 19/00

(57) ABSTRACT

Disclosed is the use of a compound with the following formula (I):

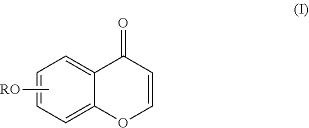

R is a linear or branched alkyl group including from 2 to 20 carbon atoms, the compound being in the form of a racemic mixture, an enantiomer (R) or an enantiomer (S), as an attractant or repellent for blood-sucking, biting invertebrates.

The present application concerns chromone derivatives as attractants and repellents of blood-sucking, biting invertebrates, in particular mosquitoes. The present application further concerns compositions which are attractant or repellent for blood-sucking, biting invertebrates.

Insects play an important role in the ecological balance. Insects are responsible for most plant pollination and are an important part of the food chain. But some insects are harmful (pest) for crops or even dangerous as vectors of infectious agents for humans and animals. For example, mosquitoes are responsible for most cases of transmission of pathogens which cause infectious diseases. In particular, the species Aedes albopictus is a vector of a plurality of arboviruses such as Dengue, Chikungunya, yellow fever and Zika. The most used control method for controlling mosquito populations is the application of insecticides which can be effective but raise problems of significant pollution because of the toxicities thereof, the broad spectra of action thereof which can kill non-target populations (useful and auxiliary insects). In addition, the appearance of insecticide resistance in vector species makes the approach generally incompatible with sustainable development. The use of repellents for protecting exposed populations or attractants for the construction of specific traps appears increasingly as supplements and/or alternatives to the use of insecticides. Relatively effective repellents such as DEET, picaridin and PMD already exist, but such molecules often exhibit side effects (eye and skin irritations, headaches, respiratory problems) and/or limited efficacy from the point of view of protection time. There are also known attractants (isovaleric acid, 3-octenol or carbon dioxide, etc.), but same do not have selectivity towards mosquitoes and have short activity durations (Andrianjafy thesis. 2018, 119,120p-«Ecologie chimique, une alternative de lutte pour le contrôle des moustiques (Diptera: Culicidae) vecteurs de maladies: bioessai, synthèse et évaluation sur terrain des répulsifs et attractifs» [“Chemical ecology, an alternative control for mosquito control (Diptera: Culicidae) Disease vectors: Bioassay, synthesis and field evaluation of repellents and attractants”]. Doctoral thesis at the Doctoral School Valorization of Renewable Natural Resources of the University of Antananarivo). The problem of the duration of activity is important and such parameter is linked to the high volatility of the products used, which are liquids with a high vapor pressure. Coumarins are a family of molecules with low vapor pressure with either repellent properties or attractant properties (Andrianjafy thesis. 2018, 110-122p; Andrianjafy et al. 2018). Such molecules are effective but to obtain sufficiently high and selective repellent or attractant effects, it is generally necessary to use mixtures of attractants or repellents (Thesis Andrianjafy 2018, 120-121p; Andrianjafy M T, Ravaomanarivo L H, Vestalys Ramanandraibe V, Rakotomanga M F, Mavingui P, Lemaire M (2018) Synthesis, bioassays and field evaluation of hydroxycoumarins and their alkyl derivatives as repellents or kairomones for Aedes albopictus Skuse (Diptera: Culicidae). J Chem Ecol 44(3):299-311; Becker N M, Zgomba D P, and Ludwig M (1995) Comparison of carbon dioxide, octanol and o a houst-odour as mosquito attractants in the Upper Valley, Germany. Med Vet Entomol 9: 377-380; Hall D R, Beevor P S, Cork A, Nesbitt B F and Vale G A (1984) 1-octen-ol: a potent olfactory stimulant and attractant for tsetse isolated from cattle odors. Insect Sci Appl 5:335-339; Shone S M, Ferrao P N, Lesser C R, Glass G E and Norris D E (2003) Evaluation of carbon dioxide and 1-octen-3-ol-baited centers for disease control Fay-Prince traps to collect Aedes albopictus. J Am Mosq Control Assoc 19:445-447; Govere J, Durrheim D N, Du Toit N, Hunt R H (2000) Local plants as repellents against Anopheles arabiensis, in Mpumalanga Province, South Africa. Cent Afr J Med 46(8):213-6; Barnard D R (2004) Laboratory evaluation of mosquito repellents against Aedes albopictus, Culex nigripalpus, and Ochlerotatus triseriatus (Diptera: Culicidae). Journal of Med Entomol 41:726-730). Moreover, in nature, insects also use mixtures of molecules (semiochemical compounds) for interacting with the environment thereof, with each other and with other living beings. Hence, in order to prepare repellent formulations (sprays, creams, mosquito nets, clothing, etc.) and attractant formulations (lures, selective traps), respectively, for limiting host-vector contact and for controlling target insect populations, it is necessary to search for new molecules apt to interact selectively with dangerous insects.

The goal of the present invention is to provide a family of compounds having attractant or repellent properties for blood-sucking, biting invertebrates, in particular for mosquitoes.

Another goal of the present invention is to provide attractant or repellent compositions for blood-sucking, biting invertebrates, in particular for mosquitoes.

Thereby, the present invention relates to the use of a compound with the following formula (I):

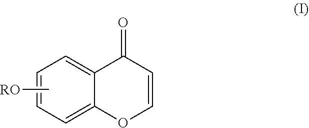

R being a linear or branched alkyl group comprising from 2 to 20 carbon atoms,
said compound being achiral when R is a linear group or in the form of a racemic mixture, an enantiomer (R) or an enantiomer (S) when R is a branched group, as attractant or repellent for blood-sucking, biting invertebrates.

The term “blood-sucking, biting invertebrates” refers to biting invertebrates which feed on blood, including biting insects which attack humans, livestock or domestic animals.

According to one embodiment, the blood-sucking, biting invertebrates are chosen from blood-sucking insects and mites, in particular blood-sucking insects.

In a preferred embodiment, the blood-sucking, biting invertebrates are selected from the group consisting of mosquitoes, ceratopogonidae, phlebotominae, bed bugs, fleas and ticks.

According to a preferred embodiment, the blood-sucking, biting invertebrates are chosen from mosquitoes of the genera Aedes, Anopheles and Culex.

In a preferred embodiment, the blood-sucking, biting invertebrates are mosquitoes selected from the group consisting of the species: Aedes albopictus, Aedes aegypti, Anopheles gambiae s.l, Anopheles mascarensis, Anopheles funestus, Anopheles stephensi, Culex pipiens quinquefasciatus and Culex pipiens pipiens.

According to the invention, an attractant compound for blood-sucking, biting invertebrates is a compound with attractant properties for blood-sucking, biting invertebrates, i.e. a compound which attracts blood-sucking, biting invertebrates.

According to the invention, a blood-sucking, biting invertebrates repelling compound is a compound having blood-sucking, biting invertebrates repelling properties, i.e. a compound which removes and therefore repels blood-sucking, biting invertebrates.

Within the framework of the present invention, the term “alkyl group” means a saturated, linear or branched aliphatic hydrocarbon group comprising, unless otherwise stated, from 2 to 20 carbon atoms. Examples include the groups ethyl, n-propyl, isopropyl, butyl, isobutyl, tertbutyl or pentyl, or nonyl or decyl.

According to one embodiment, in the above-mentioned formula (I), R represents a linear or branched alkyl group comprising from 4 to 20 carbon atoms.

According to one embodiment, in the above-mentioned formula (I), R represents a linear or branched alkyl group comprising from 4 to 16, in particular from 4 to 10, carbon atoms.

As indicated hereinabove, when R is a linear group in the formula (I) as defined hereinabove, the corresponding compound does not have an asymmetric carbon and is thus an achiral compound.

When R is a branched group in the formula (I) as defined hereinabove, the corresponding compound is then in the form of a racemic mixture, an enantiomer (R) or an enantiomer (S).

According to one embodiment, the compounds used according to the present invention correspond to the following formula (1-1):

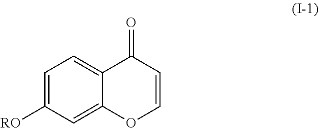

R being as defined hereinabove for the formula (I).

The present invention further relates to the use of a compound with the following formula (II):

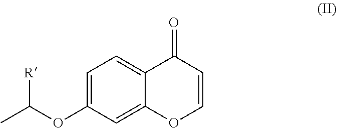

R being a linear alkyl group comprising from 2 to 18 carbon atoms, said compound being in the form of a racemic mixture, an enantiomer (R) or an enantiomer (S), as an attractant or repellent for blood-sucking, biting invertebrates.

The present invention further relates to the above-mentioned use of a compound with the formula (III):

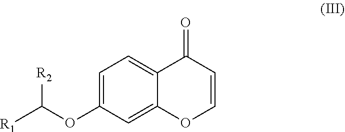

wherein:
either R₁ is H or a methyl group and R₂ is an alkyl group comprising 3 to 18 carbon atoms,
either R₂ is H or a methyl group and R₁ is an alkyl group comprising 3 to 18 carbon atoms, as an attractant for blood-sucking, biting invertebrates, said compound is in the form of a racemic mixture, of an enantiomer (R) or of an enantiomer (S).

The present invention further relates to the above-mentioned use of a compound with the formula (II):

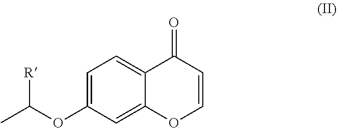

wherein R′ represents an alkyl group containing 3 to 18 carbon atoms, as an attractant for blood-sucking, biting invertebrates,
said compound is in the form of a racemic mixture, of an enantiomer (R) or of an enantiomer (S).

The present invention further relates to the above-mentioned use of a compound with the formula (II-1):

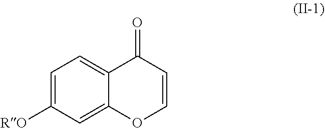

wherein R″ represents a linear alkyl group containing 4 to 20 carbon atoms, as an attractant for blood-sucking, biting invertebrates,
said compound is in the form of a racemic mixture, of an enantiomer (R) or of an enantiomer (S).

The present invention further relates to the use of a compound with the formula (II) as defined hereinabove, wherein R′ represents an alkyl group comprising 2 carbon atoms, as a repellent for blood-sucking, biting invertebrates, said compound being in the form of a racemic mixture, of an enantiomer (R) or of an enantiomer (S).

According to one embodiment, the compound with the formula (II) for the above-mentioned use is in the form of the enantiomer (R) or of a racemic mixture.

According to one embodiment, the compound with the formula (II) for the above-mentioned use is in the form of the enantiomer (S), and the compound is used in a composition wherein the content of said compound is greater than or equal to 20% by weight with respect to the total weight of said composition.

The present invention further relates to the aforementioned use of a compound with the formula (II) as an attractant for blood-sucking, biting invertebrates, wherein R′ represents an alkyl group comprising 2 carbon atoms, said compound being in the form of the enantiomer (S), and wherein the compound is used in a composition wherein the content of said compound is less than or equal to 20% by weight with respect to the total weight of said composition.

As compounds with the formula (I) used according to the invention, the following compounds can be mentioned in particular: 7-sec-butoxychromone; 7-sec-pentoxychromone; 7-sec-nonyloxychromone; 7-R-(−)-sec-butoxychromone; 7-S-(+)-sec-butoxychromone; R-(−)-sec-pentoxychromone; 7-n-decyloxychromone and 7-(2′-ethyl)hexyloxychromone.

The present invention further relates to compositions comprising the aforementioned compounds with the formula (I), (II), (III) or (II-1), having attractant or repellent properties.

The present invention further relates to an attractant or repellent composition for blood-sucking, biting invertebrates, comprising at least one compound with the formula (I) as defined hereinabove, said compound being in the form of a racemic mixture, of an enantiomer (R) or of an enantiomer (S),
said composition optionally further comprising an additional compound which is attractant or repellent for a blood-sucking, biting invertebrate.

The present invention further relates to an attractant composition for blood-sucking, biting invertebrates as defined hereinabove, comprising:
at least one compound with the formula (I) as defined hereinabove, wherein R represents an alkyl group comprising from 2 to 16 carbon atoms, preferentially from 5 to 10 carbon atoms, or
at least one compound with the formula (I) as defined hereinabove, wherein R represents an alkyl group comprising 4 carbon atoms, said compound being in the form of the enantiomer (S), and wherein the compound in the form of the enantiomer (S) is used in a concentration less than or equal to 20% of said composition.

The present invention further relates to an attractant composition for blood-sucking, biting invertebrates as defined hereinabove, comprising:
at least one compound with the formula (II) as defined hereinabove, wherein R′ represents an alkyl group comprising from 3 to 18 carbon atoms, preferentially from 3 to 10 carbon atoms, or
at least one compound with the formula (II) as defined hereinabove, wherein R′ represents an alkyl group comprising 2 carbon atoms, said compound being in the form of the enantiomer (S), and wherein the compound in the form of the enantiomer (S) is used in concentration less than or equal to 20% of said composition.

The present invention further relates to a repellent composition for blood-sucking, biting invertebrates as defined hereinabove, comprising:
at least one compound with the formula (I) wherein R represents an alkyl group containing 4 carbon atoms, the compound with the formula (I) being in the form of the enantiomer (R) or of a racemic mixture, or of a mixture of the enantiomers (R) and (S) in mass concentrations different from 50/50%; or
at least one compound with the formula (I) wherein R represents an alkyl group comprising 4 carbon atoms, said compound being in the form of the enantiomer (S) and wherein said compound in the form of the enantiomer (S) is used in a concentration greater than or equal to 1% by weight with respect to the weight of said composition.

The present invention further relates to a repellent composition for blood-sucking, biting invertebrates as defined hereinabove, comprising:
at least one compound with the formula (II) wherein R′ represents an alkyl group containing 2 carbon atoms, the compound with the formula (I) being in the form of the enantiomer (R) or a racemic mixture, or of a mixture of the enantiomers (R) and (S) in mass concentrations different from 50/50%; or
at least one compound with the formula (II) wherein R′ represents an alkyl group comprising 2 carbon atoms, said compound being in the form of the enantiomer (S) and wherein said compound in the form of the enantiomer (S) is used in a concentration greater than or equal to 1% by weight with respect to the weight of said composition.

According to one embodiment, the repellent compositions according to the invention are in the form of a spray or cream, a support, e.g. a textile support or nets, or cartridges for diffusers.

According to one embodiment, the compositions of the invention are compositions which are attractant or repellent for mosquitoes selected from the group consisting of the species: Aedes albopictus, Aedes aegypti, Anopheles gambiae s.l, Anopheles mascarensis, Anopheles funestus, Anopheles stephensi, Culex pipiens quinquefasciatus and Culex pipiens pipiens.

The compositions of the invention can further comprise an additional compound which is attractant or repellent for a blood-sucking, biting invertebrate. Such combination further improves the attractant or repellent properties of the composition of the invention.

For example, an attractant composition of the invention can comprise the attractant molecule 4-hydroxy-coumarin, in combination with a compound with the formula (I) or (II), (II-1) or (III) as defined hereinabove. Such a combination is synergistic and improves the attractant properties of the compound (I), (II), (II-1) or (III) compared with a composition comprising only one compound (I), (II), (II-1) or (III).

The present invention further relates to a kit for trapping a blood-sucking, biting invertebrates, in particular mosquitos, and in particular Aedes albopictus, comprising:
at least one attractant composition as defined hereinabove, and
at least one trap for blood-sucking, biting invertebrates, in particular mosquitos, and more particularly Aedes albopictus.

The traps used according to the invention are the traps well known to a person skilled in the art.

Traps include particular the sentinel trap BG (Biogent®) or the light trap CDC for catching Aedes sp. And Anopheles sp., respectively.

The present invention further relates to the use of the aforementioned kit for trapping blood-sucking, biting invertebrates, in particular mosquitoes, more particularly Aedes albopictus and Anopheles sp.

The present invention further relates to a repellent support for blood-sucking, biting invertebrates, in particular for mosquitoes, and more particularly Aedes albopictus, said support being impregnated with at least one repellent composition as defined hereinabove, said support being chosen in particular from the group consisting of textiles, more particularly mosquito nets, clothing for hiking, bracelets, necklaces or cartridges for diffusers.

Such a repellent support is prepared by impregnating a support as defined hereinabove with a repellent composition according to the invention, as defined hereinabove. The impregnation step can be carried out e.g. by spraying or soaking.

### FIGURES

FIG. 1 represents the synergistic effect between the compounds 4-hydroxycoumarin and 7-nonyloxychromone with regard to Aedes albopictus. Said figure represents the IK (in %) as a function of the amount deposited (in mg). The curve with diamonds corresponds to 4-hydroxycoumarin, the curve with squares corresponds to 7-nonyloxychromone and the curve with triangles corresponds to the synergy.

FIG. 2 represents the IK (in %) as a function of the amount deposited (in mg) for 7-n-decyloxychromone with regard to Aedes albopictus.

FIG. 3 represents the IK (in %) as a function of the amount deposited (in mg) for 7-(2′-ethyl)hexyloxychromone with respect to Aedes albopictus.

### EXAMPLES

### Equipment and Methods

### Synthesis of Chromone Derivatives

7-hydroxy-4-chromone is used as the starting substance for obtaining the ether derivatives. The synthesis is carried out according to the effect of the structure of the molecule on the behavior of mosquitoes.

1. Synthesis by phase transfer catalysis

A reaction with a phase transfer catalyst is used for the synthesis of non-chiral substances as shown below:

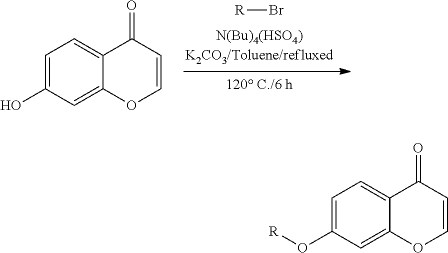

### Example 1: Synthesis of Racemic 7-sec-butoxychromone (1CPT)

100 ml of toluene and 8.4 g of K₂CO₃ (61 mmol), are added to a 250 ml three-neck reflux reactor equipped with a magnetic stirrer and a thermometer, then 2 g of 7-hydroxychromone (12 mmol) and 10.14 g of 2-bromobutane (82 mmol) are added to the mixture. For the phase transfer catalyst, 0.1 g of tetrabutyl ammonium hydrogen sulfate is added. The mixture is stirred under reflux for 6 h at 120° C. and the organic phase is recovered by decanting, washed with distilled water, dried with Na₂SO₄ and then evaporated. The resulting oily mixture is purified by column chromatography with silica gel (eluent: ethyl acetate 2 hexane 8). The pure product is obtained in the form of a yellow oil with a yield equal to 73%.

### Example 2: Synthesis of Racemic 7-sec-pentoxychromone (2CPT)

100 ml of toluene and 4.2 g of K₂CO₃ (30.6 mmol), are added to a 250 ml three-neck reflux reactor equipped with a magnetic stirrer and a thermometer, then 1 g of 7-hydroxychromone (6.15 mmol) and 5 g of 2-bromopentane (41.2 mmol) are added to the mixture. For the phase transfer catalyst, 0.05 g of tetrabutyl ammonium hydrogen sulfate is added. The mixture is stirred under reflux for 6 h at 120° C. and the organic phase is recovered by decanting, washed with distilled water, dried with Na₂SO₄ and then evaporated. The oily mixture is purified by column chromatography with silica gel (eluent: ethyl acetate 2 hexane 8). The pure product is obtained in the form of a yellow oil with a yield equal to 33.1%.

### Example 3: Synthesis of Racemic 7-sec-nonyloxychromone (3CPT)

100 ml of toluene and 2.13 g of K₂CO₃ (15.3 mmol), are added to a 250 ml three-neck reflux reactor equipped with a magnetic stirrer and a thermometer, then 0.5 g of 7-hydroxychromone (3.1 mmol) and 3 g of 2-bromononane (20.6 mmol) are added to the mixture. For the phase transfer catalyst, 0.025 g of tetrabutyl ammonium hydrogen sulfate is added. The mixture is stirred under reflux for 6 h at 120° C. and the organic phase is recovered by decanting, washed with distilled water, dried with Na₂SO₄ and then evaporated. The oily mixture is purified by column chromatography with silica gel (eluent: ethyl acetate 2 hexane 8). The pure product is obtained in the form of a yellow oil with a yield equal to 35.2%.

### Example 4: Synthesis of 7-isopropyloxychromone (4CPT)

7 ml of toluene and 0.8 g of K₂CO₃ (5.8 mmol), 0.25 g of 7-hydroxychromone (1.54 mmol) are added to a 15 ml sealed tube equipped with magnetic stirrer and a thermometer, then 2 g of 2-bromopropane (16.26 mmol) are added to the mixture. As phase transfer catalyst, 0.4 g of tetrabutyl ammonium hydrogen sulfate is added. The mixture is stirred for 24 hours at 90° C. and the organic phase is recovered by decanting, washed with distilled water, dried with Na₂SO₄ and then evaporated. The oily mixture is purified by column chromatography with silica gel (eluent: ethyl acetate 2 hexane 8). The pure product is obtained in the form of white solids with a yield equal to 29.4%.

### 2. Synthesis by the Mitsunobu Reaction

The Mitsunobu reaction is used for selectively converting an alcohol into different functional groups such as ether, in only one step. Such reaction is also used for a chiral inversion of enantiomers of chiral alcohols, as shown below:

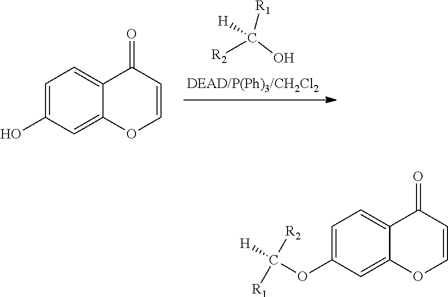

### Example 5: Synthesis of R-(−)-sec-butoxychromone (5M)

15 ml of dichloromethane, 1 g of 7-hydroxychromone (6.2 mmol), then 1.2 ml of S-butan-2-ol (12.4 mmol) and 3.2 g of triphenylphosphine (12.4 mmol) are successively added to a 50 ml reactor equipped with a thermometer, a magnetic stirrer and a slight nitrogen overpressure. The mixture is then stirred for a few minutes and then 2.2 ml of DEAD (diethyl azodicarboxylate) solution (24 mmol) are added dropwise. The reaction is carried out at room temperature for 15 to 24 hours. The solution is then evaporated and the mixture is purified by column chromatography with silica gel (eluent: ethyl acetate 2 hexane 8). The pure product is obtained in the form of a pale yellow oil with a yield equal to 52.6%.

### Example 6: Synthesis of S-(+)-sec-butoxychromone (6M)

30 ml of dichloromethane, 2 g of 7-hydroxychromone (12.4 mmol), then, 2.4 ml R-butan-2-ol (24.6 mmol) and 6.4 g of triphenylphosphine (24.6 mmol) are successively added to a 50 ml reactor equipped with a thermometer, a magnetic stirrer and a slight nitrogen overpressure. The mixture is then stirred for a few minutes and then 4.2 ml of DEAD solution (24 mmol) are added dropwise. The reaction is carried out at room temperature for 15 to 24 hours. The solution is then evaporated and the mixture is purified by column chromatography with silica gel (eluent: ethyl acetate 2 hexane 8). The pure product is obtained in the form of a pale pink oil with a yield equal to 53%.

### Example 7: Synthesis of (R) 7-sec-pentoxychromone (7M)

30 ml of dichloromethane, 1 g of 7-hydroxychromone (6.2 mmol), then 1.4 ml of S-pentan-2-ol (12.3 mmol) and 3.23 g of triphenylphosphine (12.3 mmol) are successively added to a 50 ml reactor equipped with a thermometer, a magnetic stirrer and a slight nitrogen overpressure. The mixture is then stirred for a few minutes and then 2.1 ml of DEAD solution (12.3 mmol) are added dropwise. The reaction is carried out at room temperature for 15 to 24 hours. The solution is then evaporated and the mixture is purified by column chromatography with silica gel (eluent: ethyl acetate 2 hexane 8). The pure product is obtained in the form of a pale pink oil with a yield equal to 63.3%.

### Example 8: Synthesis of (S) 7-sec-pentoxychromone (8M)

30 ml of dichloromethane, 1 g of 7-hydroxychromone (6.2 mmol), then 1.4 ml R-pentan-2-ol (12.3 mmol) and 3.23 g of triphenylphosphine (12.3 mmol) are successively added to a 50 ml reactor equipped with a thermometer, a magnetic stirrer and a slight nitrogen overpressure. The mixture is then stirred for a few minutes and then 2.1 ml of DEAD solution (12.3 mmol) are added dropwise. The reaction is carried out at room temperature for 15 to 24 hours. The solution is then evaporated and the mixture is purified by column chromatography with silica gel (eluent: ethyl acetate 2 hexane 8). The pure product is obtained in the form of a pale yellow oil with a yield equal to 42.4%.

### Example 4bis: Synthesis of 7-n-decyloxychromone (5CPT)

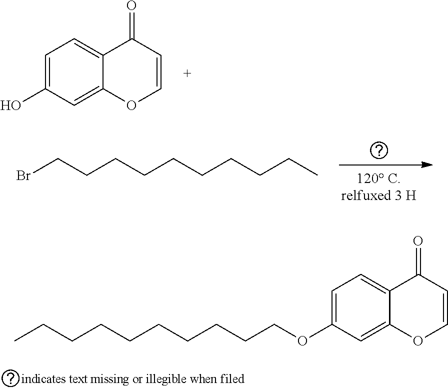

10 ml of toluene and 0.691 g of K₂CO₃ (5 mmol), are added to a 50 ml three-neck reflux reactor equipped with a magnetic stirrer and a thermometer, then 0.2 g of 7-hydroxychromone (1 mmol) and 0.660 g of 1-bromodecane (3 mmol) are added to the mixture. For the phase transfer catalyst, 0.05 g of tetrabutyl ammonium hydrogen sulfate is added. The mixture is stirred under reflux for 3 h at 120° C. and the organic phase is recovered by decanting, washed with distilled water, dried with Na₂SO₄ and then evaporated. The oily mixture is purified by extraction by liquid-liquid partitioning (hexane and distilled water). The pure product is obtained in the form of a white solid (MP=45° C.) with a yield equal to 66%.

### Example 4ter: Synthesis of 7-(2′-ethyl)hexyloxychromone

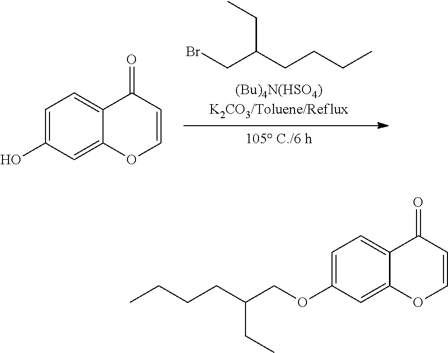

10 ml of toluene and 0.691 g of K₂CO₃ (5 mmol), are added to a 50 ml three-neck reflux reactor equipped with a magnetic stirrer and a thermometer, then 0.2 g of 7-hydroxychromone (1.2 mmol) and 0.580 g of 1-bromo-2-ethylhexane (3 mmol) are added to the mixture. For the phase transfer catalyst, 0.02 g of tetrabutyl ammonium hydrogen sulfate is added. The mixture is stirred under reflux for 6 h at 120° C. and the organic phase is recovered by decanting, washed with distilled water, dried with Na₂SO₄ and then evaporated. The crude mixture is purified by column chromatography with silica gel (eluent: hexane/ethyl acetate 98/2). The pure product is obtained in the form of a yellow oil with a yield equal to 65%.

### CHEMICAL ANALYSIS

The RMN ¹H and ¹³C spectra of the synthesized products are obtained with a BRUKER spectrometer.

An ATAGO POLAX-D polarimeter is used for determining the optical rotations of chiral products with a 2 dm observer tube. The products are diluted in 0.2% ethanol.

### EVALUATION OF THE REPELLENT AND/OR ATTENUATING EFFECT OF CHROMONE ETHERS IN A TUNNEL OLFACTOMETER

The tunnel olfactometer is a 1 m long glass parallelepiped with a 5×5 cm side. The tube is equipped with 3 openings: two on each side, one for feeding in the paper impregnated with the product to be tested, and the other for the paper containing a control. The opening in the middle is for feeding in mosquitoes. Both ends of the tube are closed by covers.

The olfactometer is divided into three compartments or zones:
a neutral zone in the middle;
a test or treated area which is on the side of depositing the product; and
a control area which is located on the side of depositing the control.

For each test, 15 Aedes albopictus females (aged 5 to 12 days) are fed into the neutral zone of the tunnel olfactometer. Mosquitoes are kept for 10 minutes for a time of adaptation. Papers impregnated and dried with a solution of product in ethanol and pure ethanol as a reference are fed in, then the barriers are opened to allow mosquitoes to circulate freely inside the tube. Three test repetitions were carried out for each product. A repetition lasts 20 minutes and the results observed were recorded every 5 minutes.

The values measured are the activity index and the repulsion index. The activity index (Al) describes the percentage of mosquitoes in the control zone (T) and in the treated zone (P) with respect to the total number of mosquitoes tested. Said value should be greater than 30% for the test to be considered significant.

The repulsion index (RI) represents the percentage of the difference in the number of mosquitoes in the control part and in the treated part divided by the sum of the two values. A negative value of the parameter (RI) indicates an attractant activity (kairomone index. KI) of the product tested.

$$\begin{matrix}AI = ( \frac{T + P}{15} ) \times 100 & RI = ( \frac{T - P}{T + P} )\end{matrix} \times 100$$

Al: Activity index
RI: Repulsion index
T: Number of mosquitoes in the control area
P: Number of mosquitoes in the treated area

### RESULTS

### Synthesis of Chromone Derivatives

### 1. Synthesis by Phase Transfer Catalysis

Five products were synthesized as a function of the length of the alkyl chain: 7-sec-butoxychromone, 7-sec-pentoxychromone, 7-sec-nonyloxychromone, 7-isopropyloxychromone and 7-decyloxychromone. The results obtained are presented in Table 1.

TABLE 1
Results obtained from the synthesis of non-chiral chromone
ethers by the phase transfer catalysis reaction

| Input | Halogenoalkane | Product obtained | Pure yield | Appearance |
|---|---|---|---|---|
| 1 CPT | 2-bromobutane | 7-sec-butoxychromone | 73% | Yellow oil |
| 2 CPT | 2-bromopentane | 7-sec-pentoxychromone | 33.1% | Yellow oil |
| 3 CPT | 2-bromononane | 7-sec-nonyloxychromone | 35.2% | Yellow oil |
| 4 CPT | 2-bromopropane | 7-isopropyloxychromone | 29.4% | White solid |
| 5 CPT | 1-bromodecane | 7-n-decyloxychromone | 66% | Yellow solid |

### 2. Synthesis by the Mitsunobu Reaction

Four enantiomers were synthesized: R-(−)-sec-butoxychromone, S-(+)-sec-butoxychromone, R-(−)-sec-pentoxychromone and S-(+)-sec-pentoxychromone. Table 2 shows the results obtained during the synthesis reactions.

TABLE 2
Results obtained from the synthesis of chiral
products by the Mitsunobu reaction

| | | | Optical | | |
|---|---|---|---|---|---|
| | | | rotation | | |
| | Chiral | | (0.2% in | Pure | |
| Input | alcohol | Product obtained | EtOH) | yield | Appearance |
| 5 M | (S) | R-(−)-sec- | −35 | 52.6% | Pale yellow |
| | butan- | butoxychromone | | | oil |
| | 2-ol | | | | |
| 6 M | (R) | S-(+)-sec- | +35 | 53% | Pale pink oil |
| | butan- | butoxychromone | | | |
| | 2-ol | | | | |
| 7 M | (R) | R-(−)-sec- | −22.5 | 63.3% | Pale pink oil |
| | pentan- | pentoxychromone | | | |
| | 2-ol | | | | |
| 8 M | (S) | S-(+)-sec- | +22.5 | 42.4% | Pale pink oil |
| | pentan- | pentoxychromone | | | |
| | 2-ol | | | | |

### CHEMICAL ANALYSIS OF PRODUCTS BY NUCLEAR MAGNETIC SPECTROMETRY (NMR)

Racemic 7-sec-butoxychromone (1CPT)

RMN ¹H (300 MHZ) in CDCl₃: 8.11 (1H, d, H₅), 7.77 (1H, d, H₂), 6.96 (1H, dd, H₆), 6.82 (1H, s, H₈), 6.26 (1H, d, H₃), 4.41 (1H, m, H₁₁), 1.77 (2H, m, H₁₃), 1.36 (3H, d, H₁₂), 1 (3H, t, H₁₄)

RMN¹³C in CDCl₃: 177.05, 162.90, 158.31, 154.83, 127.18, 118.40, 115.49, 112.83, 101.82, 75.82, 28.99, 9.68

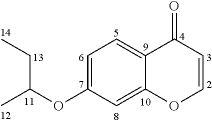 [SMILES: CCC(C)Oc1ccc2c(=O)ccoc2c1]

Yield=73% (pale yellow oil) 7-sec-pentoxychromone (2CPT) RMN ¹H (300 MHZ) in CDCl₃: 8.13 (1H, d, H₅), 7.75 (1H, d, H₂), 6.9 (1H, DD, H₆), 6.8 (1H, S, H₈), 6.28 (1H, d, H₃), 4.5 (1H, m, H₁₁), 1.75 (2H, m, H 13), 1.6 (3H, D, H₁₂), 1.38 (2H, M, H₁₄), 0.90 (3H, t, H₁₅)

RMN ¹³C in CDCl₃: 177.03, 162.90, 158.31, 154.79, 127.19, 118.40, 115.45, 112.84, 101.75, 74.42, 38.31, 19.44, 18.64, 13.95

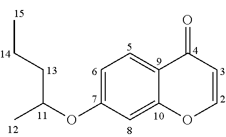 [SMILES: CCCC(C)Oc1ccc2c(=O)ccoc2c1]

Yield=33.1% (pale yellow oil)
7-sec-nonyloxychromone (3CPT)

RMN ¹H (300 MHZ) in CDCl₃: : 8.09 (1H, d, H₅), 7.75 (1H, d, H₂), 6.93 (1H, dd, H₆), 6.8 (1H, s, H₈), 6.28 (1H, d, H₃), 4.46 (1H, m, H₁₁), 1.76 (2H, m, H₁₃), 1.62 (3H, d, H₁₂), 1.41 (2H, m, H₁₈), 1.34 (2H, t, H₁₇), 1.34 (2H, t, H₁₆), 1.28 (2H, t, H₁₅), 1.28 (2H, t, H₁₄), 0.87 (3H, t, H₁₉) RMN¹³C in CDCl₃: 177, 162.88, 158.30, 154.77, 127.18, 118.40, 115.44, 112.84, 101.77, 74.69, 36.18, 31.75, 29.46, 29.18, 25.40, 22.61, 19.45, 14.05

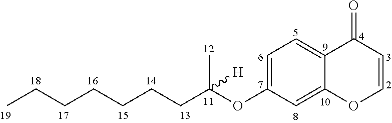 [SMILES: CCCCCCCC(C)Oc1ccc2c(=O)ccoc2c1]

Yield=35.2% (pale yellow oil)
7-isopropyloxychromone (4CPT)

RMN ¹H (300 MHZ) in CDCl₃:8.09 (1H, d, H₅), 7.76 (1H, d, H₂), 6.93 (1H, dd, H₆), 6.8 (1H, s, H₈), 6.26 (1H, d, H₃), 4.64 (1H, m, H₁₁), 1.35 (3H, d, H₁₂), 1.35 (3H, d, H₁₃),

RMN¹³C in CDCl₃: 176.98, 162.52, 158.27, 154.77, 127.15, 118.43, 115.43, 112.84, 101.81, 70.73, 21.77

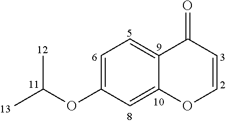

Yield=29.4% (White solid)
7-decyloxychromone

RMN ¹H (300 MHZ) in CDCl₃: 8.02 (1H, d, H₅), 7.68 (1H, d, H₂), 6.89 (1H, dd, H₆), 6.75 (1H, d, H₈), 6.20 (1H, d, H 3), 3.87-3.85 (2H, d, H₁), 1.72 (1H, m, H₁₂), 1.49 (2H, m, H₁₃), 1.39(2H, m, H₁₇), 1.26 (2H, m, H₁₆), 1.19 (2H, m, H₁₅), 0.90(3H, m, H₁₄), 0.85 (3H, m, H₁₈),

RMN ¹³C in CDCl₃: 177.98 (C₄), 164.24 (C₇), 158.49 (C₁₀), 155.60 (C₂), 126.79 (C₅), 118.08 (C₉), 115.32 (C₃), 112.34 (C₆), 100.78 (C₈), 71.21 (C₁₁), 39.15 (C₁₂), 30.36 (C₁₅), 28.95 (C₁₆), 23.72 (C₁₃), 22.88 (C₁₇), 13.83 (C₁₈), 10.88 (C₁₄)

7-(2′-ethyl)hexyloxychromone

RMN ¹H (300 MHZ) in CDCl₃: 8.03 (1H, d, H₅), 7.68 (1H, d, H₂), 6.84 (1H, dd, H₆), 6.75 (1H, d, Ha), 6.20 (1H, d, H₃), 3.87-3.85 (2H, d, H₁₁), 1.72 (1H, m, H₁₂), 1.49 (2H, m, H₁₃), 1.39(2H, m, H₁₇), 1.26 (2H, m, H₁₆), 1.19 (2H, m, H₁₅), 0.90(3H, m, H₁₄), 0.85 (3H, m, H₁₈),

RMN ¹³C in CDCl₃: 177.98 (C₄), 164.24 (C₇), 158.49 (C₁₀), 155.60 (C₂), 126.79 (C₅), 118.08 (C₉), 115.32 (C₃), 112.34 (C₆), 100.78 (C₈), 71.21 (C₁₁), 39.15 (C₁₂), 30.36 (C₁₅), 28.95 (C₁₆), 23.72 (C₁₃), 22.88 (C₁₇), 13.83 (C₁₈), 10.88 (C₁₄)

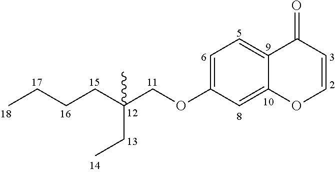 [SMILES: CCCCC(C)(CC)COc1ccc2c(=O)ccoc2c1]

### EVALUATION OF THE REPELLENT AND/OR ATTENUATING EFFECT OF CHROMONE ETHERS IN A TUNNEL OLFACTOMETER

Chromone Derivatives Having Repellent Properties Against Aedes albopictus

### Example 9: Evaluation of Racemic 7-sec-butoxychromone (1CPT)

The results obtained for the evaluation of the repellent or attractant activity against Aedes albopictus with the 7-sec-butoxychromone compound racemate are described in Table 3 for doses of products corresponding to 5, 10, 30 and 60 mg/ml.

TABLE 3
Comparison by t-test of the means of the number of mosquitoes
in the two zones: control and treated. Activity index
and repulsion index for 7-sec-butoxychromone.

| Dose | Product | Control | | | | |
|---|---|---|---|---|---|---|
| (mg/ml) | (±SD) | (±SD) | t | p-value | AI (%) | RI (%) |
| 5 | 3.3 ± 0.1 | 7.3 ± 0.4 | −12.8 | 0.0002 | 71.1 | 37.6 |
| 10 | 3.6 ± 0.4 | 7.2 ± 0.9 | −4.52 | 0.01 | 71.8 | 37.9 |
| 30 | 3.4 ± 0.9 | 8.3 ± 0.5 | −5.43 | 0.006 | 78 | 56.1 |
| 60 | 1.8 ± 0.2 | 11.8 ± 0.7 | −16.5 | <0.0001 | 90.6 | 72.9 |

### Example 10: Evaluation of R-(−)-sec-butoxychromone (5M)

The results obtained for the evaluation of the repellent or attractant activity against Aedes albopictus with the racemic compound 7-sec-butoxychromone are described in Table 4 for doses of products corresponding to 5, 10, 30 and 60 mg/ml.

TABLE 4
Comparison by t-test of the means of the number of mosquitoes
in the two zones: control and treated. Activity index
and repulsion index for R-(−)-sec-butoxychromone.

| Dose | Treated | Control | | | | |
|---|---|---|---|---|---|---|
| (mg/ml) | (±SD) | (±SD) | t | p-value | AI (%) | RI (%) |
| 5 | 3.1 ± 0.1 | 9.5 ± 0.3 | −25.12 | <0.0001 | 84 | 51.3 |
| 10 | 2.3 ± 0.7 | 11 ± 0.3 | −16.4 | <0.0001 | 88.9 | 65.6 |
| 30 | 1.4 ± 0.1 | 12.4 ± 0.1 | −93.3 | <0.0001 | 92.2 | 79.5 |
| 60 | 1.6 ± 0.2 | 12.2 ± 0.1 | −66.7 | <0.0001 | 92 | 76.6 |

### Example 11: Evaluation of S-(+)-sec-butoxychromone (6M)

The results obtained for the evaluation of the repellent or attractant activity against Aedes albopictus with the racemic compound 7-sec-butoxychromone are described in Table 5 for doses of products corresponding to 5, 10, 30 and 60 mg/ml.

TABLE 5
Comparison by t-test of the means of the number of mosquitoes
in the two zones: control and treated. Activity index
and repulsion index for S-(+)-sec-butoxychromone.

| Dose | Treated | Control | | | | |
|---|---|---|---|---|---|---|
| (mg/ml) | (±SD) | (±SD) | t | p-value | AI (%) | RI (%) |
| 5 | 9.0 ± 0.2 | 4.8 ± 0.5 | 10.4 | 0.0004 | 91.7 | −31.2 |
| 10 | 8.9 ± 1.1 | 4.2 ± 0.9 | 4.03 | 0.016 | 87.2 | −35.8 |
| 30 | 3.3 ± 0.2 | 9.3 ± 0.8 | −8.8 | 0.001 | 83.3 | 47.9 |
| 60 | 2.0 ± 0.0 | 11.4 ± 0.8 | −15.6 | <0.0001 | 89.4 | 70.5 |

Activity indices are very high >70% indicating that the results are reliable. The racemic compound and the compound (R) are repellent at any dose. The compound (S) is attractant at low doses and repellent at high doses. Such phenomenon of inversion of the effect has already been observed on some coumarins.

It is noted that the racemic compound has an effect corresponding substantially to the mean of the effects of the two enantiomers.

### Example 12: Comparison of (R) 7-sec-butoxychromone with Picaridin and DEET

The repellent properties of the (R) 7-sec-butoxychromone are now compared with same of known repellents at equivalent doses of 10 and 30 mg/ml; it is observed that the new repellent is as effective and more effective than Picaridin and DEET, which are considered to be the most effective products on the market.

TABLE 6
Comparison of the effects of (R) 7-sec-
butoxychromone with DEET and picaridin.

| | Repulsion | Repulsion | Repulsion |
|---|---|---|---|
| | index 5 mg | index 10 mg | index 30 mg |
| | deposition | deposition | deposition |
| (R) 7-sec- | 51% | 65.6% | 79.5% |
| butoxychromone | | | |
| DEET | 55.5% | 60.4% | 59.1% |
| Picaridin | 42.6% | 49.6% | 59.7% |

Chromone Derivatives Having Attractant Properties Towards Aedes albopictus

It is equally interesting to obtain attractant products for harmful insects because same can be used for the manufacture of selective and effective traps for dangerous insects. Some substituted chromones have shown attractant activities.

The syntheses of such compounds are carried out according to the same methods as for the 7-sec-butoxychromones: by phase transfer catalysis for the racemic compound and by Mitsunobu reaction for the enantiomers R and S.

### Example 13: Evaluation of racemic 7-sec-pentoxychromone or (+/−) (2CPT)

The results obtained for the evaluation of the attractant activity towards Aedes albopictus with the racemic compound 7-sec-pentoxychromone are described in Table 7 for doses of products corresponding to 1, 5, 10 and 30 mg/ml.

TABLE 7
Comparison by t-test of the means of the number of mosquitoes
in the two zones: control and treated. Activity index
and repulsion index for 7-sec-pentoxychromone.

| Dose | Treated | Control | | | | |
|---|---|---|---|---|---|---|
| (mg/ml) | (±SD) | (±SD) | t | p-value | AI (%) | KI (%) |
| 1 | 9.3 ± 0.7 | 3.5 ± 0.5 | 8.49 | 0.001 | 85 | −45.6 |
| 5 | 9.5 ± 0.2 | 2.5 ± 0.3 | 23.5 | <0.0001 | 80.1 | −59.2 |
| 10 | 9.8 ± 1.1 | 3.4 ± 0.4 | 6.8 | 0.002 | 88.3 | −47.9 |
| 30 | 8.7 ± 0.4 | 4.8 ± 0.4 | 8.5 | 0.001 | 90 | −28.6 |

### Example 14: Evaluation of (R) 7-sec-pentoxychromone (6M)

The results obtained for the evaluation of the attractant activity towards Aedes albopictus with the racemic compound (R) 7-sec-pentoxychromone are described in Table 8 for doses of products corresponding to 1, 5, 10 and 30 mg/ml.

TABLE 8
Comparison by t-test of the means of the number of mosquitoes
in the two zones: control and treated. Activity index
and repulsion index for R-(−)-sec-pentoxychromone

| Dose | Treated | Control | | | | |
|---|---|---|---|---|---|---|
| (mg/ml) | (±SD) | (±SD) | t | p-value | AI (%) | KI (%) |
| 1 | 8.6 ± 0.2 | 5.3 ± 0.3 | 10 | 0.001 | 92.2 | −24.3 |
| 5 | 10.8 ± 0.6 | 2.2 ± 0.4 | 14.7 | 0.001 | 86.7 | −66.6 |
| 10 | 8.6 ± 0.1 | 3.6 ± 0.4 | 16 | <0.0001 | 81.1 | −47.1 |
| 30 | 6.9 ± 0.3 | 6.6 ± 0.4 | 0.89 | 0.42 | 90 | −2.8 |

### Example 15: Evaluation of (S) 7-sec-pentoxychromone (7M)

The results obtained for the evaluation of the attractant activity towards Aedes albopictus with the racemic compound (S) 7-sec-pentoxychromone are described in Table 9 for doses of products corresponding to 1, 5, 10 and 30 mg/ml.

TABLE 9
Comparison by t-test of the means of the number of mosquitoes
in the two zones: control and treated. Activity index
and repulsion index for S-(+)-sec-pentoxychromone.

| Dose | Treated | Control | | | | |
|---|---|---|---|---|---|---|
| (mg/ml) | (±SD) | (±SD) | t | p-value | AI (%) | KI (%) |
| 1 | 10.3 ± 0.7 | 3.8 ± 0.3 | 10.3 | 0.0004 | 93.3 | −46.3 |
| 5 | 9.5 ± 0.7 | 3.1 ± 1.2 | 5.9 | 0.004 | 83.9 | −52.4 |
| 10 | 8.4 ± 0.1 | 5.8 ± 0.2 | 13.8 | 0.0001 | 95 | −18.2 |
| 30 | 6.5 ± 0.7 | 5.2 ± 0.3 | 2.15 | 0.09 | 77.8 | −10.7 |

The two enantiomers R, S and the racemate of 7-pentoxychromone are attractant for Aedes albopictus. The racemic effect appears to cumulate the effects of the 2 pure enantiomers.

A bell curve is obtained like the curve obtained with known kairomones (isovaleric acid, 1-octen-3-ol) (Andrianjafy et al. 2017).

### Example 16: Evaluation of 7-sec-nonyloxychromone (3CPT)

The 7-sec-nonyloxychromone is evaluated in a tunnel olfactometer for doses of 2 to 30 mg/ml. The results are shown in Table 10.

TABLE 10
Comparison by t-test of the means of the number of mosquitoes
in the two zones: control and treated. Activity index
and repulsion index for 7-sec-nonyloxychromone.

| Dose | Treated | Control | | | | |
|---|---|---|---|---|---|---|
| (mg/ml) | (±SD) | (±SD) | t | p-value | AI (%) | KI (%) |
| 0.1 | 11.2 ± 0.6 | 2.5 ± 0.2 | 19.6 | <0.0001 | 91.1 | −63.5 |
| 2 | 12.3 ± 0.0 | 1.4 ± 0.4 | 29.8 | <0.0001 | 91.1 | −79.8 |
| 5 | 10.4 ± 0.1 | 2.2 ± 0.3 | 35 | <0.0001 | 83.9 | −65.8 |
| 10 | 11.3 ± 0.0 | 2.5 ± 0.5 | 15.1 | 0.0001 | 91.7 | −63.6 |
| 30 | 8.4 ± 0.2 | 4.8 ± 0.4 | 10.4 | 0.0004 | 88.3 | −27.1 |

As for the 7-sec-pentoxychromone, a bell-shaped curve is obtained with a maximum value of KI close to 80% for a dose of 2 mg/ml. The attractant activity stays above 65% up to 10 mg/ml. Even at high doses (30 mg/ml), 7-nonyloxychromone stays attractant. And taking into account that the vapor pressure of the product should be low due to the high molecular mass thereof, the effectiveness will be long-lasting.

### Example 17: Evaluation of the Attractant Properties of 7-n-Decyloxychromone for Aedes albopictus

The results obtained for the evaluation of the attractant activity with regard to Aedes albopictus with the 7-n-decyloxychromone compound racemate are presented in Table 11 for the product dose corresponding to 10 mg/ml.

| Dose | Treated | Control | AI | KI (±SD) |
|---|---|---|---|---|
| (mg/ml) | (±SD) | (±SD) | (%) | (%) |
| 10 | 8.8 ± 0.9 | 3.9 ± 0.3 | 85 | −37.8 ± 5.2 |

7-n-decyloxychromone has an attractant effect equivalent to that of 7-sec-nonyloxychromone but has the advantage of being much less expensive (inexpensive and bio-sourced primary halide).

### Example 18: Comparison of 7-sec-pentoxychromone and 7-sec-nonyloxychromone with octen-3-ol

The attractant properties of the racemic 7-sec-pentoxychromone and 7-sec-nonyloxychromone are now compared with octen-3-ol.

TABLE 12
Comparison of 7-sec-pentoxychromone and
7-sec-nonyloxychromone with octen-3-ol

| | Repulsion | Repulsion | Repulsion |
|---|---|---|---|
| | index 5 mg | index 10 mg | index 30 mg |
| | deposition | deposition | deposition |
| 7-sec- | −59.2% | −47.8% | −28.7% |
| pentoxychromone | | | |
| 7-sec- | −65.8% | −63.7% | −27.1% |
| nonyloxychromone | | | |
| Octen-3-ol | −41.7% | −54.7% | −61.6% |

The analysis of the results on alkoxy chromones shows an attractant activity equivalent to same of octenol. However, it is known that octenol has a high vapor pressure unlike alkoxy chromones which could thus have a longer lasting effect.

The results hereinabove show that the compounds of the invention have significant attractant or repellent properties on Aedes albopictus depending on the structure of the substituent (chain length, stereochemistry, etc.).

Unlike the hydroxy or methoxy groups, the 2-butoxy groups lead to significant repellent effects depending on the stereochemistry at the 2-butoxy chain. The repellent effect of the enantiomer R is as significant as DEET and picaridin at identical doses.

Secondary alkoxy groups higher than butyl (pentyl and nonyl) lead to products of high molecular masses and having attractant properties. The attractant effect also depends on the stereochemistry of the 2 alkoxy group. Similarly, chromones with long-chain primary alkoxy substituents (e.g. decyl) have a very high attractant effect. And comparing with the most used attractant (octenol) with the same doses, alkoxychromones have a higher attractant effect.

With 7-butoxychromones, it is possible to formulate protective sprays, creams and textiles containing the products alone or mixed with other repellents.

With attractant products, traps (mechanical, glue-impregnated materials, etc.) baited with the products alone or mixed with other attractants are used for mass trapping of mosquitoes and other blood-sucking insects.

Such products derived from 7-hydroxychromone have the advantage of having high molecular masses allowing them to have a significant long-lasting effectiveness in terms of repulsion and attraction with regard to Aedes albopictus.

### Example 19: Evaluation of Repellent Properties in a Cage Olfactometer

The long-term evaluation (24 hours) is carried out in a cage system (1 m×1 m×2 m) equipped with two traps, one baited and the other is the control. The tests are carried out in parallel in two experimental rooms (35 m×2 m×2 m) which are maintained at 25+/−5° C., relative humidity 60%, for a photoperiod of 12 h (Andrianjafy et al. 2017). In the large cage is placed a small cage (35 cm×35 cm×35 cm) where the mosquitoes are placed before dropping, as well as the two traps (with the test product and the control). The traps are separated by 1.45 m. For the tests, 100 μl of the solutions are deposited on filter papers using ethanol as solvent. The ethanol is evaporated before feeding into the trap. A source of CO₂ is placed outside the cage, so as to increase the activity of mosquitoes. 25 females of Aedes albopictus are used for each test which lasts 24 hours and starts at 9 am. Six replications are carried out for each dose of a product.
Test over 24 h in a cage for the repellent properties of (R) 7-sec-butoxychromone.
Test over a period of 24 h in a cage for the attractant properties of (RS) 7-sec-nonyloxychromone.
> In natura test with a “sentinel” trap of the racemic (RS) 7-sec-nonyloxychromone.
Test of synergistic effects for the attractant properties of (RS) 7-sec-nonyloxychromone and 4-hydroxycoumarin

### Example 20: Tests with Traps

“In natura” tests of a compound or mixture according to the invention are carried out with the sentinel trap BG (Biogent®) and the light trap CDC, for catching Aedes sp. And Anopheles sp., respectively. The results obtained are conclusive.

### Example 21: Evaluation of the Combination of a Compound of the Invention with a Chromone Compound

It is possible to combine a chromone compound with other compounds of different structure. For example, 4-hydroxy-coumarin is a selective attractant of Aedes albopictus, the use of a 50/50 mixture of 4-hydroxy-coumarin and 7-(2nonyloxy)-chromone has a higher attractant power than the two compounds used separately.

7-nonyloxychromone has an attractant effect at low dose (or quantity) with a kairomone index KI-57%. A significant decrease in said effect is observed from the 10 mg dose onwards. Similarly, at low doses, 4-hydroxycoumarin also has an attractant effect with a KI on the order of 40%. A 10% increase in attraction was recorded at higher doses (10 and 30 mg).

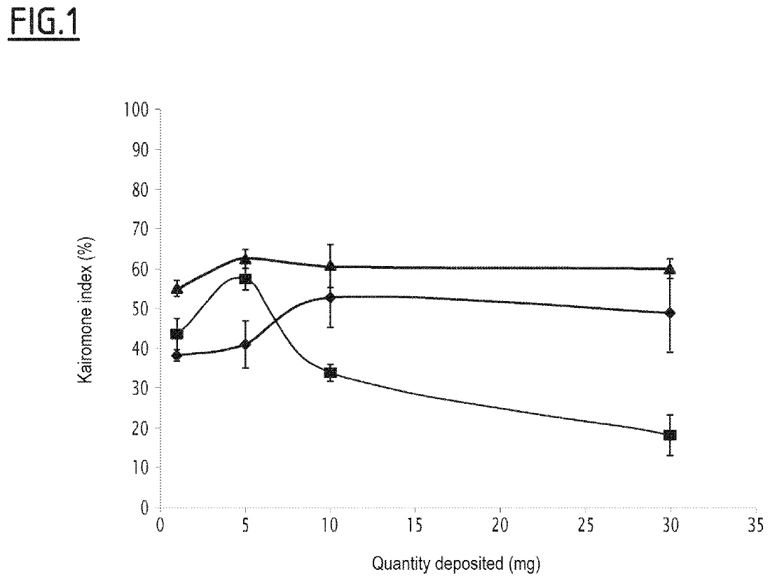

A synergistic effect is observed between 4-hydroxycoumarin and 7-nonyloxychromone (cf. FIG. 1). Such effect is generally little dependent on concentration, unlike pure products, with a KI>55% for all doses tested, which is an important practical advantage. We assume that 4-hydroxycoumarin (appearance: solid) stabilizes 7-nonyloxychromone (appearance: liquid) in order to generate a greater attractant effect.

### Example 22: Evaluation of 7-n-decyloxychromone and 7-(2′-ethyl)hexyloxychromone

The evaluation of the attractant properties of 7-n-decyloxychromone and 7-(2′-ethyl)hexyloxychromone was carried out in a tunnel olfactometer by varying the quantity of product to be tested, according to the protocol described hereinabove.

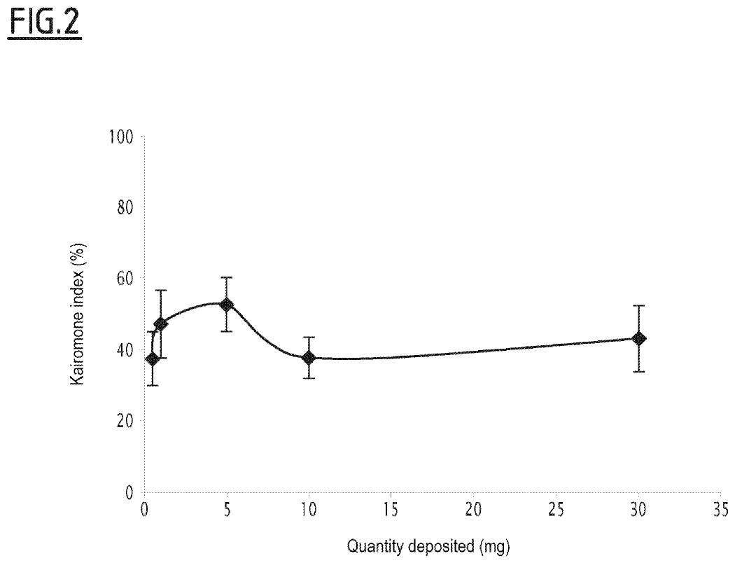

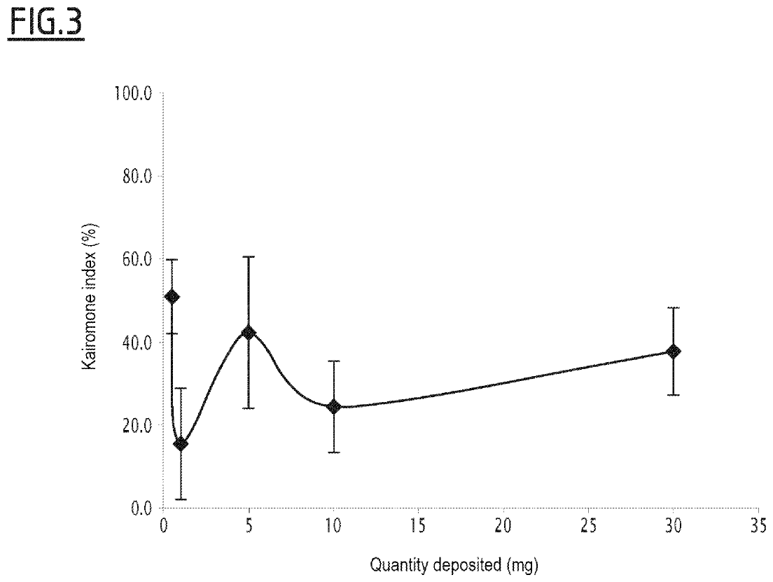

FIGS. 2 and 3 represent the results thereby obtained.

1. A method for attracting or repelling blood-sucking, biting invertebrates, comprising the use of a compound with formula (I): 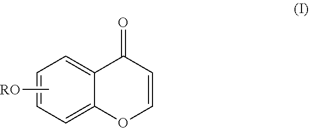
R being a linear or branched alkyl group comprising from 2 to 20 carbon atoms,
said compound being achiral when R is a linear group or in the form of a racemic mixture, an enantiomer (R) an enantiomer (S) when R is a branched group, as an.

2. The method of claim 1, for attracting blood-sucking, biting invertebrates, comprising the use of a compound with the formula (II): 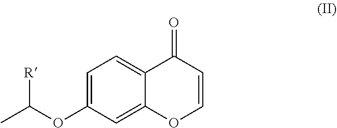
wherein R′ represents an alkyl group containing 3 to 18 carbon atoms, as an attractant for blood-sucking, biting invertebrates.

3. The method according to claim 1, for repelling blood-sucking, biting invertebrates, comprising the use of a compound with the formula (II): 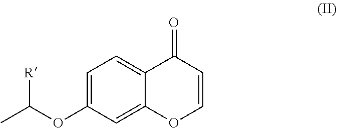
wherein R′ represents an alkyl group containing 2 carbon atoms, as a repellent for blood-sucking, biting invertebrates.

4. The method for repelling blood-sucking, biting invertebrates according to claim 3, wherein the compound with the formula (II) is in the form of the enantiomer (R) or a racemic mixture, or wherein the compound with the formula (II) is in the form of the enantiomer (S), and wherein the compound is used in a composition wherein the content of said compound is greater than or equal to 20% by weight with respect to the total weight of said composition.

5. The method for attracting blood-sucking, biting invertebrates according to claim 2, wherein R′ represents an alkyl group comprising 2 carbon atoms, said compound being in the form of the enantiomer (S), and wherein the compound is used in a composition wherein the content of said compound is less than or equal to 20% by weight with respect to the total weight of said composition.

6. An attractant or repellent composition for blood-sucking, biting invertebrates comprising at least one compound with the following formula (I): 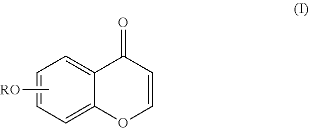
R being a linear or branched alkyl group comprising from 2 to 20 carbon atoms,
said compound being achiral when R is a linear group or in the form of a racemic mixture, an enantiomer (R) an enantiomer (S) when R is a branched group.

7. The attractant composition for blood-sucking, biting invertebrates according to claim 6, comprising:
at least one compound with the formula (I), wherein R represents an alkyl group comprising from 2 to 16 carbon atoms, or
at least one compound with the formula (I), wherein R represents an alkyl group comprising 4 carbon atoms, said compound being in the form of the enantiomer (S), and wherein the compound in the form of the enantiomer (S) is used in a concentration smaller than or equal to 20% of said composition.

8. The repellent composition for blood-sucking, biting invertebrates according to claim 6, comprising:
at least one compound with the formula (I) wherein R represents an alkyl group containing 4 carbon atoms, the compound with the formula (I) being in the form of the enantiomer (R) or of a racemic mixture, or of a mixture of the enantiomers (R) and (S) in mass concentrations different from 50/50%; or
at least one compound with the formula (I) wherein R represents an alkyl group comprising 4 carbon atoms, said compound being in the form of the enantiomer (S) and wherein said compound in the form of the enantiomer (S) is used in a concentration greater than or equal to 20% by weight with respect to the weight of said composition.

9. A kit for trapping blood-sucking, biting invertebrate, in particular Aedes albopictus, comprising:
at least a composition according to claim 6, and
at least one blood-sucking, biting invertebrate trap.

10. A method for trapping blood-sucking, biting invertebrates, comprising of providing the kit according to claim 9, and disposing the trap with the composition in the presence of the blood-sucking, biting invertebrates.

11. A repellent support for blood-sucking, biting invertebrates, said support being impregnated with at least one repellent composition according to claim 8.

12. The attractant or repellent composition of claim 6, further comprising an additional compound that is attractant or repellent for a blood-sucking, biting invertebrate.

13. The attractant composition for blood-sucking, biting invertebrates according to claim 12, comprising:
at least one compound with the formula (I), wherein R represents an alkyl group comprising from 2 to 16 carbon atoms, or
at least one compound with the formula (I), wherein R represents an alkyl group comprising 4 carbon atoms, said compound being in the form of the enantiomer (S), and wherein the compound in the form of the enantiomer (S) is used in a concentration smaller than or equal to 20% of said composition.

14. The repellent composition for blood-sucking, biting invertebrates according to claim 12, comprising:
at least one compound with the formula (I) wherein R represents an alkyl group containing 4 carbon atoms, the compound with the formula (I) being in the form of the enantiomer (R) or of a racemic mixture, or of a mixture of the enantiomers (R) and (S) in mass concentrations different from 50/50%; or
at least one compound with the formula (I) wherein R represents an alkyl group comprising 4 carbon atoms, said compound being in the form of the enantiomer (S) and wherein said compound in the form of the enantiomer (S) is used in a concentration greater than or equal to 20% by weight with respect to the weight of said composition.

15. A kit for trapping blood-sucking, biting invertebrate, comprising:
at least a composition according to claim 12, and
at least one blood-sucking, biting invertebrate trap.

16. A kit for trapping blood-sucking, biting invertebrate, comprising:
at least a composition according to claim 7, and
at least one blood-sucking, biting invertebrate trap.

17. A method for trapping blood-sucking, biting invertebrates, comprising providing the kit according to claim 15, and disposing the trap with the composition in the presence of the blood-sucking, biting invertebrates.

18. A method for trapping blood-sucking, biting invertebrates, comprising providing the kit according to claim 16, and disposing the trap with the composition in the presence of the blood-sucking, biting invertebrates.
